(19) United States
(12) Patent Application Publication
(10) Pub. No.: US 2009/0287115 A1
(43) Pub. Date: Nov. 19, 2009

(54) BUTTERFLY NEEDLE ASSEMBLY AND METHOD OF USE THEREOF

(76) Inventor: Joshua L. Abbott, Tulsa, OK (US)

Correspondence Address: HEAD, JOHNSON & KACHIGIAN, 228 W 17TH PLACE, TULSA, OK, 74119, US

(21) Appl. No.: 12/474,051

(22) Filed: May 28, 2009

Related U.S. Application Data
(63) Continuation-in-part of application No. 11/403,330, filed on Apr. 13, 2006, now abandoned.

(51) Int. Cl. A61M 5/00; A61B 5/15

(52) U.S. Cl. 600/576; 604/174; 604/180

(57) ABSTRACT

A butterfly needle assembly and method of use having a compressible member for uses such as drawing blood and infusing intravenous fluids or medication. The butterfly needle assembly also includes an intravenous needle, a body to which the compressible member is attached, a fastener for maintaining a desired position of the needle during use, an attachment mechanism and a fluid conduit in fluid communication with the intravenous needle and attachment mechanism. In use, the needle is inserted into a patient's vein. The compressible member is compressed to position the needle as desired, and the fastener is employed to secure the butterfly needle assembly to the patient in a way that maintains compression on the compressible member. The butterfly needle assembly may be further secured to the patient by an adhesive disposed on the bottom surface of the compressible member. The attachment mechanism may be attached to a container for blood collection.

### CROSS-REFERENCE TO RELATED APPLICATIONS

This application claims priority to and is a continuation-in-part of U.S. patent application Ser. No. 11/403,330, filed Apr. 13, 2006,which is incorporated herein by reference.

### BACKGROUND OF THE INVENTION

1. Field of the Invention

The invention relates generally to a butterfly needle assembly and method of use thereof. and more particularly to a butterfly needle assembly and method of use thereof uniquely configured to maintain the needle at a desired position during insertion and use.

2. Description of the Related Art

There are many uses for intravenous needles in the medical industry. Some of the more common uses of intravenous needles include administering medicine, drawing blood samples, and providing patients with intravenous fluids.

Intravenous needles are available in various configurations. So-called “butterfly” needles include wings or tabs that extend out perpendicular to the needle to help secure and steady the needle when the wings are grasped by the care provider. A small diameter hose having an attachment mechanism at one end is in fluid communication with the needle. The attachment mechanism may include a second needle for penetrating the seal on an IV fluid line or a container used to collect a blood sample. This configuration of needle assembly and attachment mechanism can be used to collect multiple blood samples by changing out the container attached to the attachment mechanism.

One of the shortcomings of prior art butterfly needles is the difficulty associated with maintaining the needle at a proper position in the patient's vein during use. Even a small amount of needle movement can place unwanted stress on the vein and cause the needle to rupture the vein or to detach from the vein. When using a butterfly needle to collect blood, the care provider must hold the needle in place with one hand while collecting blood samples with the other hand—a difficult procedure even for experienced care providers. Since the depth and location of veins below the skin vary from patient to patient, optimal positioning of the needle (particularly the angle at which the needle penetrates the skin and vein) will also vary. Unfortunately, intravenous needles available today do not effectively or efficiently maintain proper needle positioning. Instead, the position of the needle during use is dependent on the inconsistent ability of the care provider to hold the needle in place.

What is needed, therefore, is a butterfly needle assembly capable of effectively maintaining proper position of the needle during use while enabling the care provider to use both hands to attend to tasks other than holding the needle in place.

### SUMMARY OF THE INVENTION

In general, the invention relates to a butterfly needle assembly and method of use thereof that includes a needle with a central axis, a hollow interior extending along the central axis, and opposed first and second ends. A body having a top in opposed relation to a bottom is secured to the needle. A fluid conduit interconnects the second end of the needle with an attachment mechanism. A compressible member is attached to the needle assembly for being placed in a compressed state to obtain a desired position of the needle during use. A fastener is used to secure the needle assembly to the patient while the compressible member is in a compressed state so that compression of the compressible member is maintained during use.

### BRIEF DESCRIPTION OF THE DRAWINGS

FIG. 1 is a perspective view of an example of a butterfly needle assembly in accordance with an illustrative embodiment of the butterfly needle assembly and method of use thereof disclosed herein;

FIG. 2 is a perspective view of the example of the butterfly needle assembly of FIG. 1 in use in accordance with an illustrative embodiment of the butterfly needle assembly and method of use thereof disclosed herein;

FIG. 3 is a partial sectional side view of an example of a butterfly needle assembly in use on a patient with a deep vein in accordance with an illustrative embodiment of the butterfly needle assembly and method of use thereof disclosed herein;

FIG. 4 is a partial sectional side view of an example of a butterfly needle assembly in use on a patient with a shallower vein in accordance with an illustrative embodiment of the butterfly needle assembly and method of use thereof disclosed herein;

FIG. 5 is a perspective view of another example of a butterfly needle assembly in accordance with an illustrative embodiment of the butterfly needle assembly and method of use thereof disclosed herein; and

FIG. 6 is a front perspective view of the example of the butterfly needle of FIG. 5 in accordance with an illustrative embodiment of the butterfly needle assembly and method of use thereof disclosed herein.

Other advantages and features will be apparent from the following description and from the claims.

### DETAILED DESCRIPTION OF THE INVENTION

The devices and methods discussed herein are merely illustrative of specific manners in which to make and use this invention and are not to be interpreted as limiting in scope.

While the devices and methods have been described with a certain degree of particularity, it is to be noted that many modifications may be made in the details of the construction and the arrangement of the assemblies and components without departing from the spirit and scope of this disclosure. It is understood that the devices and methods are not limited to the embodiments set forth herein for purposes of exemplification.

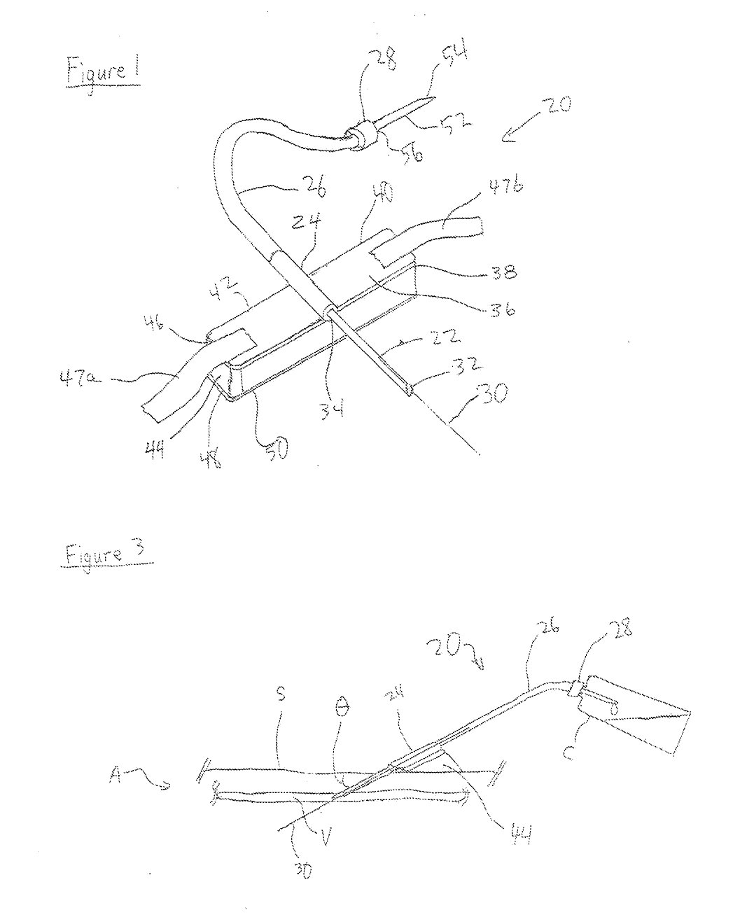

Referring to the figures of the drawings, wherein like numerals of reference designate like elements throughout the several views, and initially to FIG. 1, a butterfly needle assembly 20 includes an intravenous needle 22, a body 24, a fluid conduit 26 and an attachment mechanism 28. The intravenous needle 22 has a generally cylindrical shape with a central axis 30, a first end 32 and a second end 34 in opposed relation to the first end 32. The intravenous needle 22 has a hollow interior extending along the central axis 30.

The body 24 is secured to the needle 22 and includes a top 36 in opposed relation to a bottom 38. The general shape or footprint of the body 24 can vary greatly. As shown in FIG. 1, the body 24 has a first and second wing 40 and 42 extending perpendicular from the central axis 30 of the needle 22.

The needle 22 can be secured to the body 24 in various ways. For example and as shown in FIG. 1, the needle 22 may be friction fit to at least a portion of the body 24 where it is connected to the fluid conduit 26.

A compressible member 44 with a top surface 46 and an opposing bottom surface 48 is secured to the body 24 such that the top surface 46 of the compressible member 44 is secured to the bottom surface 38 of the body 24. The compressible member 44 is constructed from a resilient foam or other material capable of being compressed. One such foam material that may be used is visco-elastic memory foam. Other foam materials that may be used include foams made from silicon, various plastics, polyvinylchloride (PVC) and polyethylene. Other compressible materials may be used as well.

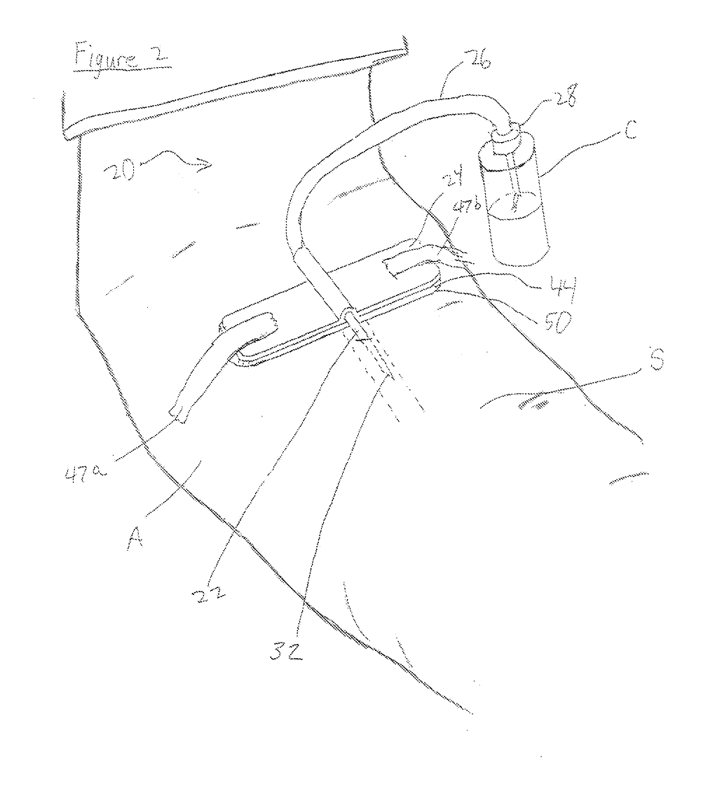

To help maintain proper positioning of the needle during use, a fastener is provided to secure the butterfly needle assembly 20 to the patient while the compressible member 44 is in a compressed state. As can be seen in FIGS. 1 and 2, the fastener may include one or more adhesive strips, or tape 47a, 47b secured to the top 36 of the body 24. After the needle 22 is inserted into the vein and the member 44 is compressed to position the needle 22 at a desired depth and angle, the butterfly needle assembly 20 is secured in place by sticking adhesive strips 47a, 47b to the patient's skin. In addition, the adhesive strips 47a, 47b include a removable film covering to protect the adhesive surfaces of the strips 47a, 47b prior to use.

If desired, a layer of adhesive 50 may be disposed on the bottom surface 48 of the compressible member 44. This layer of adhesive 50 can be used to further secure the butterfly needle assembly 20 to the skin of the patient.

The butterfly needle assembly 20 also includes an attachment mechanism 28. While the attachment mechanism 28 can take any form commonly known in the art for attaching to containers or an intravenous line, the attachment mechanism 28 may include a needle 52. The needle 52 has a first end 54 and a second end 56 with a cylindrical shape and hollow interior. The intravenous needle 22 and the attachment mechanism 28 are connected by a fluid conduit 26. The fluid conduit 26 has a hollow interior which is in fluid communication with the second end 34 of the intravenous needle 22 and the second end 56 of the attachment mechanism 28 needle 52.

FIG. 2 shows the butterfly needle assembly 20 in use on a patient to draw a blood sample. The first end 32 of the intravenous needle 22 is inserted into the arm A of a patient. The intravenous needle 22 extends through the skin S and into a vein located below the skin S in the arm A of the patient. The butterfly needle assembly 20 is first secured in place to the skin S on the arm A by an adhesive 50 exposed on the bottom surface 48 of the compressible member 44. There are also two pieces of tape 47a, 47b to secure the butterfly needle assembly 20 to the arm A once the care provider has compressed the compressible member 44 to position the needle 22 as desired. Thus, tape 47a, 47b secures the butterfly needle assembly 20 to the patient in a way that maintains compression on the compressible member 44 so as to maintain the desired position of the needle 22 during use without the care provider having to hold the butterfly needle assembly 20 in place. The blood flows through the needle 22, fluid conduit 26, and attachment mechanism 28 and into a container C.

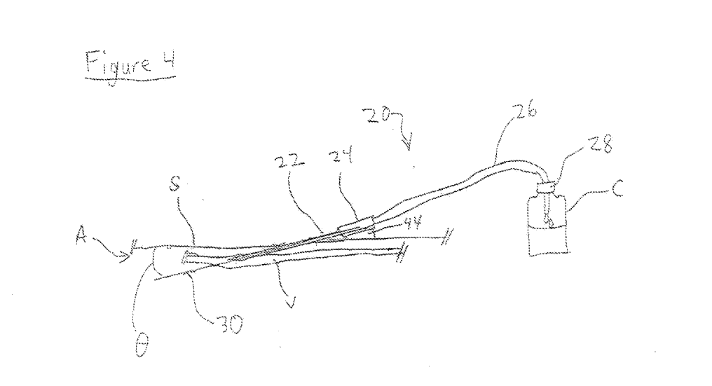

FIGS. 3 and 4 illustrate how the butterfly needle assembly 20 can be used or adapted to fit the particular vein depth during a procedure. FIG. 3 is a cross-sectional view showing the butterfly needle assembly 20 inserted into an arm A with a vein V that is relatively deep in relationship to the skin S covering the arm A. FIG. 4 provides a cross-sectional view of an arm A where the butterfly needle assembly 20 is used to obtain blood from a relatively shallow vein V below the skin S surface of the arm A. The angle of attack θ is the angle between the skin S of the patient's arm and the central axis 30 of the intravenous needle 22. As can be seen in comparing FIGS. 3 and 4, the angle of attack θ increases with the depth that the vein V lies beneath the skin S of the arm A.

In use of the butterfly needle assembly, the needle 22 is inserted through the skin S of the patient's arm A and into a vein V at an angle of attack θ such that the needle 22 is long enough to penetrate the vein V. Once the intravenous needle 22 is inserted into the vein V, the compressible member 44 is compressed to position and support the intravenous needle 22 at the angle of attack θ. To maintain the needle 22 and body 24 at the angle of attack θ, the needle 22 and body 24 are held in place by adhesive strips 47a, 47b.

Once the needle 22 and body 24 are secured in place with adhesive strips 47a, 47b, the care provider may conveniently use both hands to attach a container C to the attachment mechanism 28 such that the vein V, intravenous needle 22, fluid conduit 26, attachment mechanism 28 and container C are all in fluid communication with one another. The higher pressure of the blood inside the vein V causes the blood to flow through the intravenous needle 22, fluid conduit 26, and attachment mechanism 28 and into the container C where it collects. If necessary, the care provider can remove the first container C from the attachment mechanism 28 and replace it with a second container such that multiple samples of blood can be drawn without having to manually hold the butterfly needle assembly 20 in place. Once the desired amount of blood has been drawn, the care provider can then remove the intravenous needle 22 from the vein V and arm A. At the same time the body 24 and the compressible member 44 are removed from the skin S of the arm A. It is then often necessary to apply pressure to the site where the needle 22 penetrated the skin S using a cotton ball or gauze bandage to control bleeding.

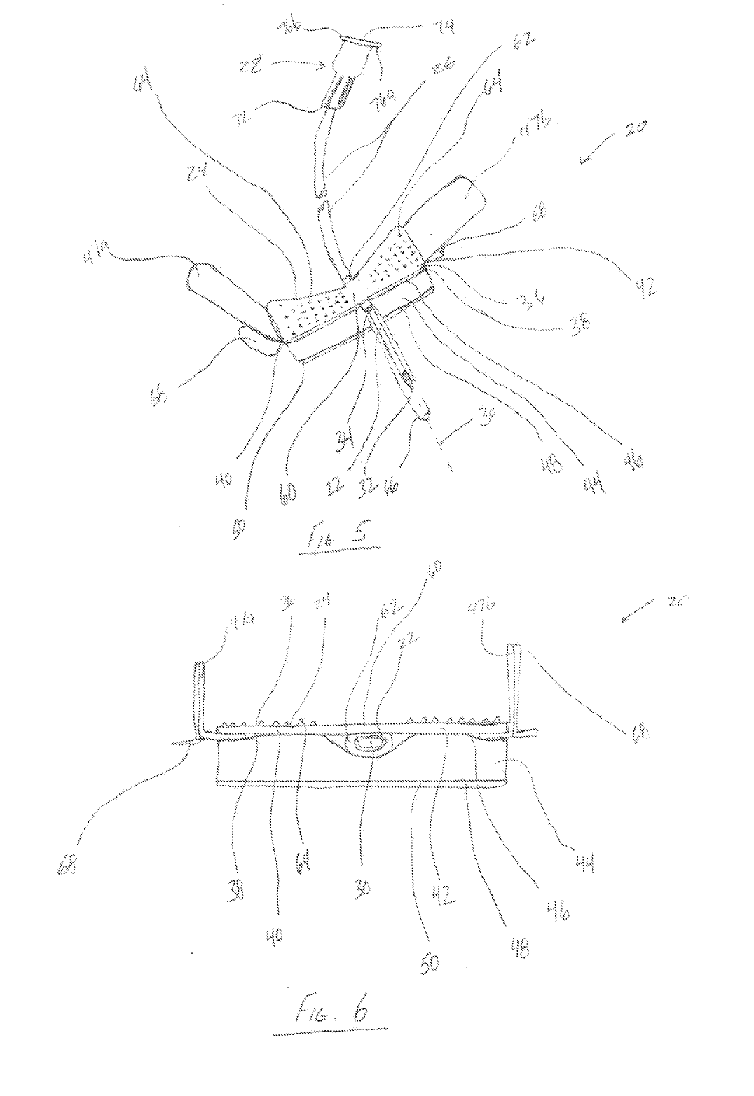

Referring now to FIGS. 5 and 6, the butterfly needle assembly 20 includes an intravenous needle 22, a body 24, a fluid conduit 26 and an attachment mechanism 28. The intravenous needle 22 has a generally cylindrical shape with a central axis 30, a first end 32 and a second end 34 in opposed relation to the first end 32. The intravenous needle 22 has a hollow interior extending along the central axis 30. The body 24 of the butterfly needle assembly 20 is secured to the needle 22 and includes a top surface 36 in opposed relation to a bottom surface 38. The top surface 36 of the body 24 may include a plurality of raised protrusions 64. The general shape or footprint of the body 24 can vary greatly. As shown in FIG. 5, the body 24 has a first and second wing 40 and 42 extends from the central axis 30 of the needle 22. As shown in FIG. 5, the first and second wing 40 and 42 may taper from a central portion 60 of the body 24 such that the terminal portions of the first and second wing 40 and 42 have a greater surface area than the surface area at the central portion 60 of the body 24. In particular, the body 24 may taper rearwardly (i.e., away from the needle 22) from the central portion 60 to the first and second wing 40 and 42, thereby not hindering placement of the needle 22 during usage.

The needle 22 can be secured to the body 24 in various ways. For example and as shown in FIGS. 5 and 6, the body 24 may include a generally cylindrical channel 62 formed as part of the bottom surface 38 along the central portion 60. The cylindrical channel 62 may protrude from the central portion 60 of the body 24 rearwardly (i.e., away from the needle 22) where it may be secured to the fluid conduit 26 and forwardly (i.e., toward the needle 22) where it may be secured to the needle 22. For example, an interior surface of the fluid conduit 26 may be friction fit to an exterior surface of the cylindrical channel 62 while an exterior surface of the needle 22 may be friction fit to an interior surface of the cylindrical channel 62, thereby placing the needle 22 in fluid communication with the fluid conduit 26. Additionally, the butterfly needle assembly 20 may include a generally cylindrical shaped needle protector 66 to protect against inadvertent piercing by the needle 22 prior to usage. An interior surface of the needle protector 66 may be friction fit to the exterior surface of the cylindrical channel 62 enabling the needle protector 66 to be easily removed prior to usage.

A compressible member 44 with a top surface 46 and an opposing bottom surface 48 is secured to the body 24 such that the top surface 46 of the compressible member 44 is secured to the bottom surface 38 of the body 24. The compressible member 44 is constructed from a resilient foam or other material capable of being compressed. One such foam material that may be used is visco-elastic memory foam. Other foam materials that may be used include foams made from silicon, various plastics, polyvinylchloride (PVC) and polyethylene. Other compressible materials may be used as well.

To help maintain proper positioning of the needle during use, a fastener is provided to secure the butterfly needle assembly 20 to the patient while compressible member 44 is in a compressed state. As can be seen in FIGS. 5 and 6, the fastener may include one or more adhesive strips, or tape 47a, 47b secured to the bottom surface 38 of the body 24, intermediate of the top surface 46 of the compressible member 44. After the needle 22 is inserted into the vein and the member 44 is compressed to position the needle 22 at a desired depth and angle, the butterfly needle assembly 20 is secured in place by sticking adhesive strips 47a, 47b to the patient's skin S. In addition, the adhesive strips 47a, 47b include a removable film 68 covering to protect the adhesive surfaces of the strips 47a, 47b prior to use. In addition, a layer of adhesive 50 may be disposed on the bottom surface 48 of the compressible member 44. This layer of adhesive 50 can be used to further secure the needle assembly 20 to the skin S of the patient.

The butterfly needle assembly 20 also includes an attachment mechanism 28 secured to the fluid conduit 26. While the attachment mechanism 28 can take any form commonly known in the art for attaching to a container C or an intravenous line, the attachment mechanism 28 may be elongate and generally cylindrical with a hollow interior, a first end 72 secured to the fluid conduit 26 and a second end 74 capable of being connected, either directly or indirectly, to a container C or an intravenous line. The needle 22 and the attachment mechanism 28 are connected in fluid communication by the fluid conduit 26. The second end 74 of the attachment mechanism 28 may include a radially projecting shoulder or a pair of outwardly projecting tabs 76a and 76b enabling the attachment mechanism 28 to be releasably secured to the container C or the intravenous line, such as by threading the projecting tabs 76a and 76b into internal threads of the container or the intravenous line.

While this application has discussed the use of a butterfly needle assembly 20 in terms of drawing blood samples, the same apparatus could be used in a similar manner as a catheter or port for providing a patient with intravenous solutions or medication.

Whereas, the devices and methods have been described in relation to the drawings and claims, it should be understood that other and further modifications, apart from those shown or suggested herein, may be made within the spirit and scope of this invention.

What is claimed is:

1. A butterfly needle assembly, comprising:
a body having a top surface in opposed relation to a bottom surface, said body having opposed left and right wings extending generally perpendicularly from a central portion of said body, said left and right wings of said body being rearwardly tapered such that a terminal portion of said left and right wings has a greater surface area than said central portion of said body, said bottom surface of said central portion of said body including a generally cylindrical channel;
a needle having a central axis, a hollow interior extending along a central axis, said needle having a first end and a second end in opposed relation to the first end, said needle secured to a first end of said generally cylindrical channel of said body;
a compressible member attached to said butterfly needle assembly for being placed in a compressed state to obtain a desired position of said needle during use, said compressible member having a top surface in opposed relation to a bottom surface, said top surface of said compressible member being secured to said bottom surface of said body, said compressible member being fabricated from a material that slowly recovers its original shape after compression;
a fastener for securing the butterfly needle assembly to the patient. while said compressible member is in a compressed state, said butterfly needle assembly being secured by said fastener in a way that maintains compression on said compressible member, said fastener being secured to said butterfly needle assembly intermediate of said bottom surface of said body and said top surface of said compressible member;
a fluid conduit secured to a second end of said generally cylindrical channel of said body; and
an attachment mechanism secured to said fluid conduit.

2. The butterfly needle assembly of claim 1 wherein said generally cylindrical channel of said body protrudes from said central portion of said body rearwardly and is secured to said fluid conduit, and wherein said generally cylindrical channel of said body protrudes from said central portion of said body forwardly and is secured to said needle.

3. The butterfly needle assembly of claim 2 wherein an interior surface of said fluid conduit is secured to an exterior surface of said cylindrical channel and an exterior surface of said needle is secured to an interior surface of said cylindrical channel.

4. The butterfly needle assembly of claim 1 further comprising a generally cylindrical shaped needle protector having an interior surface secured to an exterior surface of said cylindrical channel.

5. The butterfly needle assembly of claim 1 wherein said fastener comprises a first piece of tape secured to said bottom surface of said left wing of said body intermediate of said top surface of said compressible member and a second piece of tape secured to said bottom surface of said right wing of said body intermediate of said top surface of said compressible member.

6. The butterfly needle assembly of claim 1 wherein said fastener comprises a removable film covering to protect an adhesive surface prior to use.

7. The butterfly needle assembly of claim 1 wherein said compressible member is fabricated from foam.

8. The butterfly needle assembly of claim 7 wherein said foam is a visco-elastic foam or a polyvinylchloride foam.

9. The butterfly needle assembly of claim 1 further comprising an adhesive disposed on said bottom surface of said compressible member for further securing said butterfly needle assembly to the patient.

10. The butterfly needle assembly of claim 1 wherein said attachment mechanism includes a needle having a cylindrical shape and a hollow interior, a first end and a second end, said first end in fluid communication with said fluid conduit.

11. The butterfly needle assembly of claim 1 wherein said attachment mechanism is elongate and generally cylindrical with a hollow interior, and wherein said attachment mechanism has a first end secured to said fluid conduit and a second end capable of being secured to a container or an intravenous line.

12. The butterfly needle assembly of claim 11 wherein said second end of said attachment mechanism includes a radially projecting shoulder enabling said attachment mechanism to be releasably secured to said container or said intravenous line.

13. The butterfly needle assembly of claim 11 wherein said second end of said attachment mechanism includes a pair of outwardly projecting tabs enabling said attachment mechanism to be releasably secured to said container or said intravenous line.

14. A butterfly needle assembly, comprising;
a body having a top surface in opposed relation to a bottom surface, said body having opposed left and right wings extending generally perpendicularly from a central portion of said body, said left and right wings of said body being rearwardly tapered such that a terminal portion of said left and right wings has a greater surface area than said central portion of said body, said bottom surface of said central portion of said body including a generally cylindrical channel, said generally cylindrical channel having a first end and a second end, said first end and said second end of said generally cylindrical channel protruding in opposing directions from said central portion of said body;
a needle having a central axis, a hollow interior extending along a central axis, said needle having a first end and a second end in opposed relation to the first end, said needle secured to said first end of said generally cylindrical channel of said body;
a compressible member attached to said butterfly needle assembly for being placed in a compressed state to obtain a desired position of said needle during use, said compressible member having a top surface in opposed relation to a bottom surface, said top surface of said compressible member being secured to said bottom surface of said body, said compressible member being fabricated from a material that slowly recovers its original shape after compression;
a fastener for securing the butterfly needle assembly to the patient while said compressible member is in a compressed state, said butterfly needle assembly being secured by said fastener in a way that maintains compression on said compressible member, said fastener including a first piece of tape secured to said bottom surface of said left wing of said body intermediate of said top surface of said compressible member and a second piece of tape secured to said bottom surface of said right wing of said body intermediate of said top surface of said compressible member, wherein said first piece of tape and said second piece of tape each include a removable film covering to protect an adhesive surface thereof;
a fluid conduit secured to said second end of said generally cylindrical channel of said body; and
an attachment mechanism being elongate and generally cylindrical with a hollow interior, said attachment mechanism having a first end secured to said fluid conduit and a second end capable of being secured to a container or an intravenous line;
wherein said needle, said fluid conduit and said attachment mechanism of said butterfly needle assembly are in fluid communication.

15. The butterfly needle assembly of claim 14 further comprising a generally cylindrical shaped needle protector having an interior surface secured to an exterior surface of said cylindrical channel.

16. The butterfly needle assembly of claim 14 wherein said compressible member is a visco-elastic foam or a polyvinylchloride foam.

17. A method of using a butterfly needle assembly, said method comprising the steps of:
providing a butterfly needle assembly, said butterfly needle assembly comprising:
a body having a top surface in opposed relation to a bottom surface, said body having opposed left and right wings extending generally perpendicularly from a central portion of said body, said left and right wings of said body being rearwardly tapered such that a terminal portion of said left and right wings has a greater surface area than said central portion of said body, said bottom surface of said central portion of said body including a generally cylindrical channel, said generally cylindrical channel having a first end and a second end, said first end and said second end of said generally cylindrical channel protruding in opposing directions from said central portion of said body;
a needle having a central axis, a hollow interior extending along a central axis, said needle having a first end and a second end in opposed relation to the first end, said needle secured to said first end of said generally cylindrical channel of said body;
a compressible member attached to said butterfly needle assembly for being placed in a compressed state to obtain a desired position of said needle during use, said compressible member having a top surface in opposed relation to a bottom surface, said top surface of said compressible member being secured to said bottom surface of said body, said compressible member being fabricated from a material that slowly recovers its original shape after compression;
a fastener for securing the butterfly needle assembly to the patient while said compressible member is in a compressed state, said fastener including a first piece of tape secured to said bottom surface of said left wing of said body intermediate of said top surface of said compressible member and a second piece of tape secured to said bottom surface of said right wing of said body intermediate of said top surface of said compressible member, wherein said first piece of tape and said second piece of tape each include a removable film covering to protect an adhesive surface thereof;
a fluid conduit secured to said second end of said generally cylindrical channel of said body; and
an attachment mechanism being elongate and generally cylindrical with a hollow interior, said attachment mechanism having a first end secured to said fluid conduit and a second end capable of being secured to a container or an intravenous line;
positioning said bottom surface of said compressible member against the skin of a patient;
inserting said first end of said needle through said skin of said patient and into a vein;
compressing said compressible member to obtain a desired position of said needle for use; and
securing said butterfly needle assembly to said patient with said first piece of tape and said second piece of tape in a way that maintains compression on said compressible member.

18. The method of claim 17 further comprising the steps of:
providing a container;
securing said attachment mechanism to said container such that said container is in fluid communication with said attachment mechanism, said fluid conduit, said needle and said vein; and
collecting blood in said container.

19. The method of claim 17 further comprising the step of providing an adhesive disposed on said bottom surface of said compressible member to further secure said butterfly needle assembly to said patient.
